(19) United States
(12) Patent Application Publication
(10) Pub. No.: US 2003/0236572 A1
(43) Pub. Date: Dec. 25, 2003

(54) Total joint replacements using magnetism to control instability

(76) Inventor: Morton Bertram III, Naples, FL (US)

Correspondence Address: John G. Posa, Gifford, Krass, Groh, Sprinkle,, Anderson & Citkowski, P.C., 280 N. Old Woodward Ave., Suite 400, Birmingham, MI, 48009-5394, US

(21) Appl. No.: 10/427,060

(22) Filed: Apr. 30, 2003

Related U.S. Application Data
(63) Continuation-in-part of application No. 10/087,052, filed on Oct. 18, 2001, now abandoned.
(60) Provisional application No. 60/241,401, filed on Oct. 18, 2000.

(51) Int. Cl. A61F 2/30

(52) U.S. Cl. 623/18.12

(57) ABSTRACT

Magnetic force fields are used to control the instability of joint-replacement situations. Prosthetic components according to the invention are fabricated with opposite-polarity magnets on either side of the joint surface, so that an inherent stability is conferred to the joint. The magnets are of sufficient strength so that dislocation or uncoupling of the components would be very difficult, but not impossible. The forces do, however, allow motion between the bearing surfaces, without increasing friction between the joint surfaces. The approach is applicable to various artificial joint situations, including the hip, shoulder, ankle, elbow, knee, and smaller joints.

### REFERENCE TO RELATED APPLICATION

This application is a continuation-in-part of U.S. patent application Ser. No. 10/087,052, filed Oct. 18, 2001, which claims priority from U.S. Provisional Patent Application Serial No. 60/241,401, filed Oct. 18, 2000, the entire content of both applications being incorporated herein by reference.

### FIELD OF THE INVENTION

This invention relates generally to prosthetic components and, in particular, to total joint replacements using magnetism to control instability.

### BACKGROUND OF THE INVENTION

Total joint replacement has become a common procedure in the United States and elsewhere in the world. Arthroplasty of the hip, knee, shoulder, ankle, and elbow are the most frequent applications. Smaller joints are replaced as well.

Unfortunately, instability continues to be a problem. The most common reasons for instability of joint replacements is muscular weakness, which makes the joint less stable and more prone to dislocation or uncoupling. Other reasons include decreases in mental acuity, malposition of components, and alcohol.

It is estimated that $75 million is now spent in the United States annually in conjunction with total hip replacement instability. This includes costs associated with repeat surgeries and hospitalizations to correct instability problems. Surgical methods for controlling instability are not entirely effective. The current solution is bracing, repeat surgery to correct any malposition of components, and muscle advancement or retensioning.

A certain percentage of patients have unsolvable problems, necessitating drastic measures to address their situations. This usually involves performing a Girdlestone procedure, which involves removal of the prosthesis altogether, leaving nothing in the joint. Frequently this results in a “flail” limb, with significant functional deficits. An inability to solve these problems, not infrequently, leads to litigation because of the frustration felt by the patient. These, in turn, lead to additional costs, exascerbating the problem.

Although certain inventions have been disclosed and patented wherein magnetism is used in joint-replacement surgery, none so far have been specifically directed to solving the problems associated with instability. U.S. Pat. No. 5,879,386, entitled “Magnetic Prosthetic System” uses magnetism to hold the bones apart during articulation to reduce friction. U.S. Pat. No. 5,571,195 to Johnson, entitled “Prosthesis For An Artificial Joint Having A Wear Particle Collection Capability” utilizes magnetism to collect metal wear particles. U.S. Pat. No. 5,092,320 to Maurer uses magnets (70) to secure a knee brace to the leg of a wearer. U.S. Pat. No. 4,216,548 to Kraus utilizes magnets and electromagnetism to stimulate bone growth/ingrowth. U.S. Pat. No. 3,140,712, entitled “Articulated Joint,” for example, artificially duplicates the vacuum or suction [of a joint] by means of a magnetizable metal cup.

U.S. Pat. Nos. 4,743,264 and 4,781,720 to Sherva-Parker use magnetic traction to retain external prosthetic devices, i.e., amputation apparatus. U.S. Pat. No. 5,062,855 to Rincoe teaches the use of magnetism to control an artificial limb. U.S. Pat. No. 5,507,835 to Jore discloses a first embodiment wherein magnetic fixtures are used to hold an external prosthesis in place, and a second embodiment wherein repelling magnetic forces are used to hold bones apart.

European patents EP0578969A3 and EP0578969B1 disclose magnet arrangements for a prosthesis in particular, for a dental prosthesis. In addition, European patents EP0533384A1 and EP0533384B1 disclose a prosthesis for use with an ossicular chain to allow a magnetic to be coupled to the ossicular chain. European patents EP0638293A1 and EP0638293B1 show a device for positioning a magnet in a prosthesis, apparently a dental prosthesis as well.

### SUMMARY OF THE INVENTION

The instant invention solves problems evident in the current art by employing magnetic force fields to control the instability of joint-replacement operations. According to the preferred embodiment, prosthetic components according to the invention are fabricated with opposite-polarity magnets on either side of the joint surface, so that an inherent stability is conferred to the joint. The magnets are of sufficient strength so that dislocation or uncoupling of the components would be very difficult, but not impossible. The forces would, however, allow motion between the bearing surfaces, without increasing friction between the joint surfaces. The approach is applicable to various artificial joint situations, including the hip, shoulder, ankle, elbow, knee, and smaller joints.

### BRIEF DESCRIPTION OF THE DRAWINGS

FIG. 1 is an exploded view of a prior-art total hip system, showing how the components are assembled;

FIG. 2 is a drawing which shows how the head of a prior-art femoral component fits on the trunion of the prosthesis;

FIG. 3 is a drawing which shows a prior-art acetabular component with an apical hole;

FIG. 4 is a drawing which illustrates the components of a priora-art shoulder replacement;

FIG. 5A depicts the initial separation associated with the so-called “pistoning effect,” wherein, during the swing phase of gait, hip joint components briefly separate;

FIG. 5B depicts the heel-strike rim contact stage associated with the pistoning effect;

FIG. 5C depicts static relocation associated with the so-called pistoning effect;

FIG. 6 is a cross-sectional view of a hip system constructed in accordance with a preferred embodiment of the invention; and

FIG. 7 is a cross-sectional view of a hip system constructed in accordance with a preferred embodiment of the invention.

### DETAILED DESCRIPTION OF THE INVENTION

The invention will now be described in detail with reference to the accompanying figures. As discussed above, although the embodiments will be described in conjunction with a total hip replacement, it will be apparent to those of skill that the approach is applicable to alternate joint situations, including the shoulder, elbow, knee, and smaller joints.

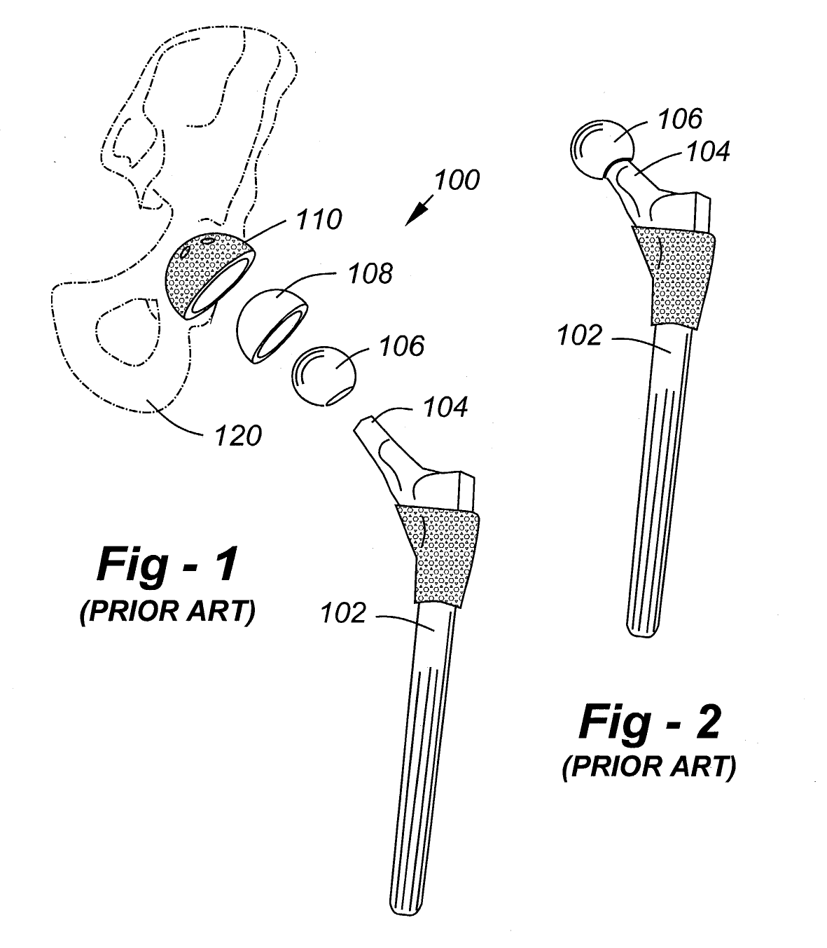

FIG. 1 is an exploded view of a typical prior-art total hip system 100, showing how the components are assembled. In this configuration, an acetabular insert 110 is inserted into the pelvis 120 following appropriate reaming or other preparation. The cup 110 can be cemented or cementless, in which case some type of porous or boney ingrowth surface is generally provided. A liner 108 fits into the cup 110, which may be of a polymer such as polyethylene, or, more modernly, a ceramic. Metal-on-metal systems are also available, any of which are applicable to the instant invention as described in further detail below.

On the femoral side of the system, an endoprosthesis having a stem 102 and a neck 104 is provided, allowing differently-sized ball-shape heads 106 to the neck 104, as shown in FIG. 2.

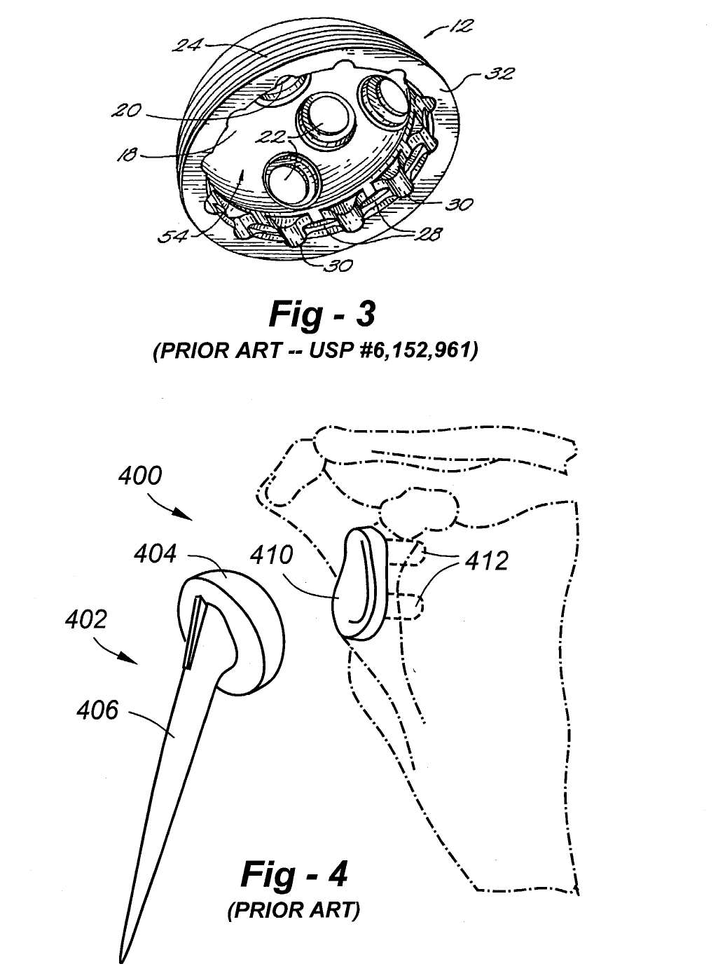

FIG. 3 is a perspective view of a prior-art acetabular shell 12. Relevant to the instant invention, such a shell may include a plurality of holes 22 extending therethrough, including an apical hole 20 for seating a bone screw.

FIG. 4 is a drawing of a typical prior-art shoulder system, depicted generally at 400. Such a system includes a humeral component 402, including a stem 406 and head 404, the latter cooperating with a glenoid replacement surface 410 seated into the bone typically using one or more posts 412.

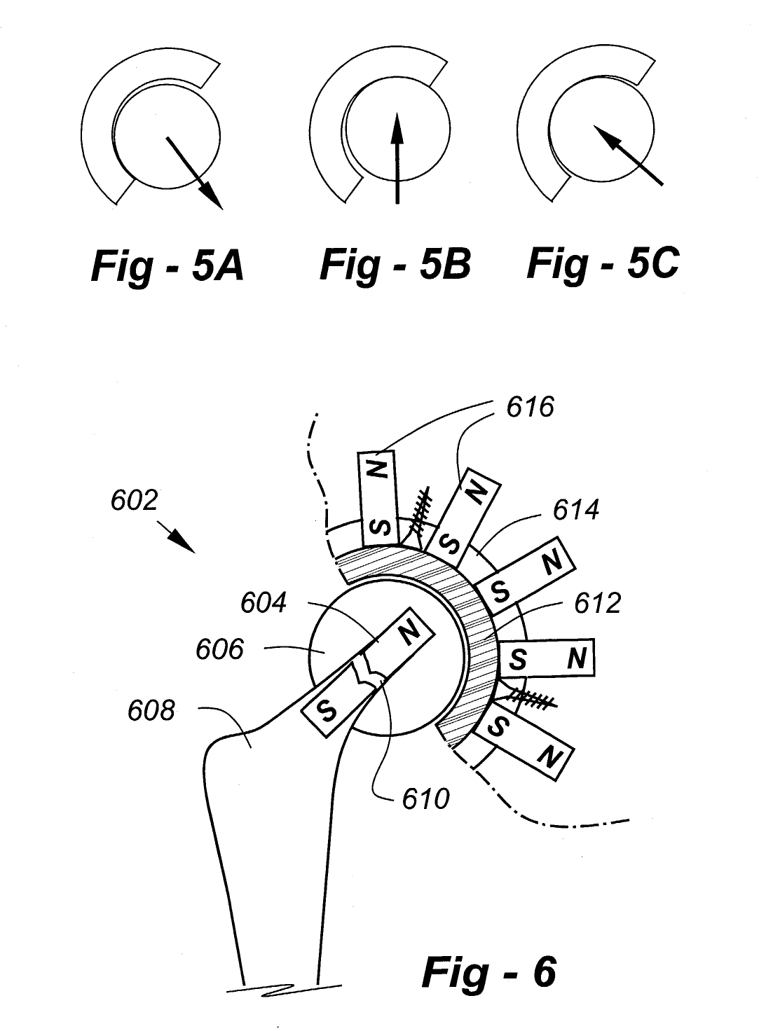

FIGS. 5A through 5C concern the so-called “pistoning effect,” wherein, during the swing phase of gait, certain joint components briefly separate. When the components recouple or “relocate,” the effect tends to increase the wear of the surfaces. It is believed that his phenomenon accounts for the fact that higher wear rates are seen in vivo, as compared to in vitro studies. FIG. 5A depicts the initial separation associated with the so-called “pistoning effect,” wherein, during the swing phase of gait, hip joint components briefly separate. FIG. 5B depicts the heel-strike rim contact stage associated with the pistoning effect, and FIG. 5C depicts static relocation associated with the pistoning effect.

FIG. 6 is a drawing which shows a preferred embodiment of the invention applied to a total hip system, depicted generally at 602. One the femoral side, a stem 608 attaches to a head 606 through a neck, and within the neck, there is disposed a magnet 604. As with the other embodiments described herein, the magnet is based upon a very high-flux-density material, preferably those made out of the rare earth group of elements; for example, neodymium-iron-boron or samarium-cobalt systems may be used. The break at 610 is used to show that the magnet 604 may be of any appropriate length to provide more or less strength. Although not limited to a modular system, the invention preferably uses at least a modular head, enabling more straightforward construction and easier installation of the magnet 604.

On the acetabular side, one or, preferably, a plurality of magnets 616 are disposed through a cup 614 in facing relationship to a liner 612, which may be of any nonmagnetic material, including polymers such as polyethylene, ceramics, or non-magnetic metals such as stainless. In the preferred embodiment, the magnets 616 are arranged axially, and pointing generally toward the center of the head 606, such that as the femoral component rotates, the greatest flux density is achieved between the opposing poles of the magnets on the femoral and acetabular sides. Although the magnet 604 is shown terminating with the north pole, and the magnets 616 are shown with the south poles pointing toward the head 606, it will be appreciated that this arrangement may be reversed and have the same affect.

The are not necessarily shown to scale, and the cup 614 may be screwed in place, cemented or cementless, utilizing a porous or boney ingrowth surface. Although not necessary to the invention, the magnets 616 may be aligned through holes such as 20 and 22 shown in FIG. 3, thereby allowing the magnets 616 to be as close as possible to the liner 612 and may, in fact, be installed after the liner and cup are implanted by drilling through the holes through the cup 614.

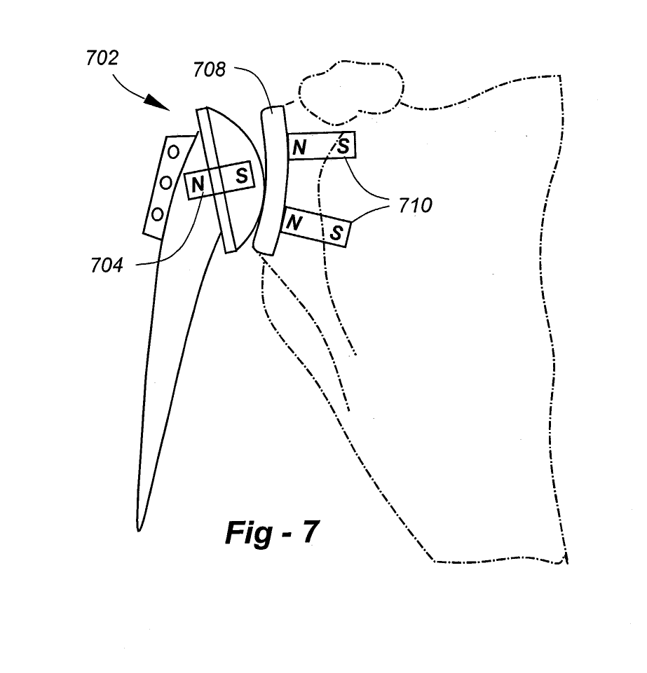

It will be appreciated that arrangement shown in FIG. 6 addresses various dislocation problems, as well as the pistoning affect described with reference to FIGS. 5A through 5C. In addition, as discussed above, the invention is not limited to the hip, but is applicable to other joints, including the shoulder, as shown in FIG. 7. Similar to the device of FIG. 6, the humeral component 702 includes at least one magnet 704, and a glenoid component 708 includes one or more magnets 710, if the polarity is reversed relative to the humeral side, thereby causing an attraction and improving stability.

The magnets according to the invention are preferably incorporated into the various components during the manufacturing process to prevent oxidation or other deterioration of the surfaces. Since magnets only work effectively within a certain range or “air gap,” beyond which the magnets exhibit no attraction, the magnets may form part of the total joint implants without fear of attraction from very strong magnetic fields of the type used with medical and industrial instrumentation. Compatibility with metal-on-metal replacements would effectively eliminate problems with the air-gap phenomenon.

I claim:

1. Reduced dislocation total joint replacement apparatus, comprising:
a first prosthetic component having a convex bearing surface;
a second component having a concave bearing surface configured to co-act with the first bearing surface;
a magnet having a magnetic polarity positioned behind the convex bearing surface; and
one or more magnets, each having an opposite polarity positioned behind the concave bearing surface,
wherein the magnetic attraction between the opposing poles minimizes dislocation or uncoupling of the components while allowing relative movement of the bearing surfaces.

2. The apparatus of claim 1, wherein the components are associated with a total hip, knee, shoulder, ankle or elbow replacement.

3. The apparatus of claim 3, wherein the components are associated with a total hip replacement, and one of the magnets is disposed in the apical hole of the acetabular component generally used for the insertion of instrumentation.
